(19) United States
(12) Patent Application Publication
(10) Pub. No.: US 2010/0069773 A1
(43) Pub. Date: Mar. 18, 2010

(54) WIRELESS PYRO/PIEZO SENSOR SYSTEM

(75) Inventors: Reinhold Henke, Plymouth, MN (US); Peter Stasz, Mounds View, MN (US)

Correspondence Address: SCHWEGMAN, LUNDBERG & WOESSNER, P.A., P.O. BOX 2938, MINNEAPOLIS, MN, 55402, US

(73) Assignee: Dymedix Corporation, Shoreview, MN (US)

(21) Appl. No.: 12/558,104

(22) Filed: Sep. 11, 2009

Related U.S. Application Data
(60) Provisional application No. 61/096,343, filed on Sep. 12, 2008.
(60) Provisional application No. 61/096,481, filed on Sep. 12, 2008.
(60) Provisional application No. 61/096,501, filed on Sep. 12, 2008.
(60) Provisional application No. 61/096,523, filed on Sep. 12, 2008.

(51) Int. Cl. A61B 5/08

(52) U.S. Cl. 600/538

(57) ABSTRACT

This document discusses, among other things, a system and method for detecting biomedical data for diagnosing sleep disorders of a patient. Biomedical data of the patient can be detected using one or more wireless sensors. The biomedical data can be processed to separate various information including respiration temperature and pressure information. The various information can be wirelessly communicated to a base station coupled to a polysomnograph (PSG) machine configured to display the information for diagnosing sleep disorders.

### PRIORITY AND RELATED APPLICATIONS

This application claims the benefit under 35 U.S.C 119(e) of the following United States Provisional Patent Applications, the contents of which are incorporated herein by reference in their entirety: U.S. Provisional Patent Application Ser. No. 61/096,343 filed Sep. 12, 2008, U.S. Provisional Patent Application Ser. No. 61/096,481 filed Sep. 12, 2008, U.S. Provisional Patent Application Ser. No. 61/096,501 filed Sep. 12, 2008, and U.S. Provisional Patent Application Ser. No. 61/096,523 filed Sep. 12, 2008.

### TECHNICAL FIELD

The present subject matter relates generally to the field of diagnosing and treating patients who suffer from sleep disorders and, more particularly, to a wireless pyro/piezo sensor system for the transmission of human biomedical data for diagnosing sleep disorders in patients at a sleep practitioner's sleep laboratory or for diagnosing and treating sleep patients at their home or other private surroundings.

### BACKGROUND

Sleep disorders have recently become the focus of a growing number of physicians. Sleep disorders include obstructive sleep apnea, central sleep apnea, complex sleep apnea, snoring, restless leg syndrome (RLS), periodic limb movement (PLM), bruxism (teeth grinding and clenching) and sudden infant death syndrome (SIDS) to name a few, and other related neurological and physiological events or conditions occurring during sleep. Many hospitals and clinics have established sleep laboratories (sleep labs) to diagnose and treat sleep disorders. In the sleep laboratories, practitioners use instrumentation to monitor and record a patient's sleep states, stages and behaviors during sleep. Practitioners rely on these recordings to diagnose patients and prescribe proper therapies.

The primary goal of addressing sleeping disorders is to help a person sleep better. The secondary goal of addressing sleeping disorders is to help a person live longer. It is well known that various undesirable behaviors often occur during sleep, such as sleep apneas, abnormal breathing, snoring, restless legs, teeth grinding and clenching and the like. It is further known that these disorders and other undesirable behaviors can not only lead to insufficient amounts of sleep or fatigue, but are also linked to co-morbidities such as obesity, high blood pressure, diabetes, cardiac diseases, stroke and SIDS, all of which lead to a pre-mature death. Serious efforts are being made to reduce or eliminate these undesirable disorders and behaviors in part because of these co-morbidity concerns.

In addressing sleep related problems, such as sleep apnea, insomnia and other physiologic events or conditions occurring during sleep, various hospitals and clinics have established laboratories, sometimes referred to as “Sleep Laboratories” (sleep labs). At these sleep labs, using instrumentation, such as wired patient bio-data sensors connected to a polysomnograph (PSG) machine, a patient's sleep patterns may be monitored and recorded via wired sensors for later analysis so that a proper diagnosis may be made and a therapy prescribed. Varieties of wired sensors have been devised for providing recordable signals related to respiratory (inhaling and exhaling) patterns during sleep. These wired sensors commonly are mechanical to electrical transducers that produce an electrical signal related to respiration.

Applicant's assignee, Dymedix Corporation of Shoreview, Minn., has pioneered the development of improved sleep medicine sensors that are adapted to be attached to the upper lip, throat area, abdomen, chest or limbs of a patient that, during sleep, produce an electric signal proportional to inspiratory and expiratory airflow, respiratory effort and to episodes of snoring.

### SUMMARY

Examples of the present subject matter provide a wireless pyro/piezo sensor system for sleep diagnostic and sleep therapy that, by means of being wireless, provides the clinical sleep practitioner and the sleep patient with a more reliable and more freedom of movement affording means of providing an un-tethered patient with sleep diagnostics testing in a sleep lab or in a home environment.

A wireless pyro/piezo sensor system, in one example, comprises a wireless pyro/piezo sensor having an integral wireless transceiver for transmitting and receiving wirelessly data and control information to and from a base station which connects and relates, via a multitude of wires, the received wireless pyro/piezo sensor information to an attached PSG machine.

In various examples, a wireless pyro/piezo sensor comprises a pyro/piezoelectric PVDF film transducer mated with an integrated or hybrid wireless pyro/piezo sensor transceiver.

The wireless pyro/piezo sensor transceiver is coupled with a radio frequency antenna, a radio frequency power detector, a wireless battery charger, a battery, a charge pump, a low power micro controller, an analog to digital converter and a wire termination means to connect to the two terminals of the pyro/piezoelectric PVDF film transducer element to the analog-to-digital converter.

A wireless pyro/piezo sensor base station according to one example may comprise a power source terminal, a power supply, such as an AC to DC power supply, a radio frequency antenna, a radio frequency transceiver coupled to the antenna, a micro controller a digital-to-analog converter, an analog signal de-multiplexer, and an analog signal filter, with means to connect a plurality of sensor information terminals to the attached PSG machine.

In Example 1, a system for detecting biomedical data for diagnosing sleep disorders of a patient includes a wireless sensor configured to detect the biomedical data from the patient, a wireless base station configured to wirelessly receive the biomedical data from the wireless sensor, and a polysomnograph (PSG) machine coupled to the wireless base station, the PSG machine configured to display information about the received biomedical data to a user for diagnosing sleep disorders.

In Example 2, the wireless sensor of Example 1 is optionally configured to produce a first output indicative of airflow temperature and to produce a second output indicative of airflow pressure.

In Example 3, the system of any one or more of Examples 1-2 optionally includes a processor coupled to the wireless base station, the processor configured to separate inspiratory and expiratory temperature change from the biomedical data, and the PSG machine is optionally coupled to the processor and configured to display the separated inspiratory and expiratory temperature change information to the user for diagnosing sleep disorders.

In Example 4, the PSG machine of any one or more of Examples 1-3 is optionally configured to display the received biomedical data from the wireless sensor and the separated inspiratory and expiratory temperature change information from the processor to the user for diagnosing sleep disorders.

In Example 5, The system of any one or more of Examples 1-4 optionally includes a processor coupled to the wireless base station, the processor configured to separate inspiratory and expiratory pressure change from the biomedical data, and the PSG machine is optionally coupled to the processor and configured to display the separated inspiratory and expiratory pressure change information to the user for diagnosing sleep disorders.

In Example 6, the PSG machine of any one or more of Examples 1-5 is optionally configured to display the received biomedical data from the wireless sensor and the separated inspiratory and expiratory pressure change information from the processor to the user for diagnosing sleep disorders.

In Example 7, the system of any one or more of Examples 1-6 optionally includes a processor coupled to the wireless base station, the processor configured to separate inspiratory and expiratory temperature change from the biomedical data and to separate inspiratory and expiratory pressure change from the biomedical data, and the PSG machine is optionally coupled to the processor and configured to display the separated inspiratory and expiratory temperature change information and the separated inspiratory and expiratory pressure change information to the user for diagnosing sleep disorders.

In Example 8, the PSG machine of any one or more of Examples 1-7 is optionally configured to display the received biomedical data from the wireless sensor and the separated inspiratory and expiratory temperature change information and the separated inspiratory and expiratory pressure change information from the processor to the user for diagnosing sleep disorders.

In Example 9, the wireless sensor of any one or more of Examples 1-8 optionally includes at least one of a respiration sensor, a chest respiratory effort belt, or an abdominal respiratory effort belt.

In Example 10, the wireless sensor of any one or more of Examples 1-9 optionally includes a respiration sensor, a chest respiratory effort belt, and an abdominal respiratory effort belt.

In Example 11, the wireless base station of any one or more of Examples 1-10 optionally includes a single wireless base station configured to wirelessly receive biomedical data from each of the respiration sensor, the chest respiratory effort belt, and the abdominal respiratory effort belt.

In Example 12, the wireless sensor of any one or more of Examples 1-11 optionally includes at least one of a tissue vibration sensor or a muscle motion sensor.

In Example 13, a method for detecting biomedical data for diagnosing sleep disorders of a patient includes detecting biomedical data of the patient using a wireless sleep sensor, wirelessly receiving the biomedical data from the wireless sleep sensor using a wireless base station coupled to a PSG machine, and displaying the sleep related information to a user using the PSG machine.

In Example 14, the detecting biomedical data of Examples 13 optionally includes detecting respiratory airflow temperature and respiratory airflow pressure of the patient using a wireless airflow sensor.

In Example 15, the method of any one or more of Examples 13-14 optionally includes processing the biomedical data to separate inspiratory and expiratory temperature change information from the biomedical data, and the displaying optionally includes displaying the separated inspiratory and expiratory temperature change information for diagnosing sleep disorders.

In Example 16, the displaying of any one or more of Examples 13-15 optionally includes displaying the received biomedical data and the separated inspiratory and expiratory temperature change information for diagnosing sleep disorders.

In Example 17, the method of any one or more of Examples 13-16 optionally includes processing the biomedical data to separate inspiratory and expiratory pressure change information from the biomedical data, and the displaying optionally includes displaying the separated inspiratory and expiratory pressure change information for diagnosing sleep disorders.

In Example 18, the displaying of any one or more of Examples 13-17 optionally includes displaying the received biomedical data and the separated inspiratory and expiratory pressure change information for diagnosing sleep disorders.

In Example 19, the method of any one or more of Examples 13-18 optionally includes processing the biomedical data to separate inspiratory and expiratory temperature change from the biomedical data and to separate inspiratory and expiratory pressure change from the biomedical data, and the displaying optionally includes displaying the separated inspiratory and expiratory temperature change information and the separated inspiratory and expiratory pressure change information to the user for diagnosing sleep disorders.

In Example 20, the displaying of any one or more of Examples 13-19 optionally includes displaying the received biomedical data, the separated inspiratory and expiratory temperature change information and the separated inspiratory and expiratory pressure change information to the user for diagnosing sleep disorders.

In Example 21, the detecting biomedical data of any one or more of Examples 13-20 optionally includes detecting at least one of chest respiratory effort information of the patient using a wireless chest respiratory effort belt, detecting abdominal respiratory effort information using a wireless abdominal respiratory effort belt, or detecting respiratory airflow information using a wireless, pyro/piezoelectric airflow sensor.

In Example 22, the detecting biomedical data of any one or more of Examples 13-21 optionally includes detecting chest respiratory effort information using a wireless chest respiratory effort belt, detecting abdominal respiratory effort information using a wireless abdominal respiratory effort belt, and detecting respiratory airflow information using a wireless pyro/piezoelectric airflow sensor.

In Example 23, the wirelessly receiving the biomedical data of any one or more of Examples 13-22 optionally includes wirelessly receiving the biomedical data from the sleep sensor using a single wireless base station coupled to a PSG machine.

In Example 24, the detecting biomedical data of any one or more of Examples 13-23 optionally includes detecting biomedical data of the patient using at least one of a wireless tissue vibration sensor or a wireless muscle motion sensor.

While the present disclosure is directed toward treatment of sleep disorders, further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

FIG. 1A illustrates generally a wireless pyro/piezo sensor system in place on a sleep diagnostic patient according to one embodiment of the present subject matter.

FIG. 1B illustrates generally a wireless pyro/piezo sensor system in place on a sleep therapy patient according to one embodiment of the present subject matter.

FIG. 2 illustrates generally an electrical block diagram of a wireless pyro/piezo sensor system according to one embodiment of the present subject matter.

FIG. 3 illustrates generally a front view of a wireless pyro/piezo sensor construction according to one embodiment of the present subject matter.

FIG. 4 illustrates generally a side view of a wireless pyro/piezo sensor construction according to one embodiment of the present subject matter.

FIG. 5 illustrates generally an electrical block diagram of the wireless pyro/piezo sensor transceiver according to one embodiment of the present subject matter.

FIG. 6 illustrates generally an assembly layout of a wireless pyro/piezo sensor transceiver according to one embodiment of the present subject matter.

FIG. 7 illustrates generally an electrical block diagram of a wireless pyro/piezo sensor base station according to one embodiment of the present subject matter.

FIG. 8 illustrates generally an assembly layout of a wireless pyro/piezo sensor base station according to one embodiment of the present subject matter.

### DETAILED DESCRIPTION

The following detailed description relates to a wireless pyro/piezo sensor system includes of a plurality of wireless pyro/piezo sensors, a plurality of wireless pyro/piezo transceivers and a single wireless pyro/piezo sensor base station also incorporating a transceiver for receiving data from the sensors and for sending message control signals to the sensors.

U.S. Pat. No. 5,311,875 to Peter Stasz first disclosed a pyro/piezoelectric sensor embodying a polyvinylidene fluoride (PVDF) film as the active element of such a respiration activity sensor.

The PVDF film has both pyroelectric and piezoelectric properties and, as such, is responsive to both inspiratory and expiratory air temperature and air pressure changes, producing a corresponding polarized electrical signal output indicating either inspiratory air temperature and pressure or expiratory air temperature and pressure. The polarized electrical sensor output signal can be processed to effectively separate the inspiratory and expiratory temperature change induced signal from the signal due to inspiratory and expiratory pressure change.

Improvements in the sensor are the subject of U.S. Pat. Nos. 6,894,427, 6,551,256, 6,485,432, 6,491,642, 6,254,545, and U.S. Pub. App. No. US2007/0012089A1, the teachings of which are hereby incorporated by reference as if set forth fully herein.

For the most part, the sensor construction described in the aforementioned patents were intended for wired PVDF film transducer based sensors in that they would sense temperature, or temperature and sound vibrations and subsequently transmit the electrical representation of the aforementioned signals via copper wires to a PSG machine. The wiring may limit the ability of the sleep subject to roll from one side to another. The wiring may also limit the patient from visiting the restroom at will.

Hence, there is a need to provide a wireless pyro/piezo sensor system that does not require the patient to be wired to the PSG head box for the duration of the sleep study.

Furthermore, there is a need to provide a wireless pyro/piezo sensor system, which collects the patient data from multiple wireless pyro/piezo sensors placed on a single patient without electrically interfering with one another.

Furthermore, there is a need to provide a wireless pyro/piezo sensor system, which collects the patient data from multiple wireless pyro/piezo sensors placed on a single patient without causing cross-talk or other interference with wireless systems being used on other sleep patients in the sleep lab facility.

Furthermore, there is a need to provide a wireless pyro/piezo sensor system, which indicates, via the output polarity of its signal at the PSG, that the respiratory signal changes are the result of either inspired or expired air movement.

Furthermore, there is a need to provide a wireless pyro/piezo sensor system, which wirelessly transmits respiratory airflow signals from the patient to the PSG.

Furthermore, there is a need to provide a wireless pyro/piezo sensor system, which wirelessly transmits respiratory effort signals from the patient to the PSG.

Furthermore, there is a need to provide a wireless pyro/piezo sensor system, which wirelessly transmits from the patient to the PSG the chest and abdominal sum signal.

Furthermore, there is a need to provide a wireless pyro/piezo sensor system, which wirelessly transmits tissue vibration signals from the patient to the PSG.

Furthermore, there is a need to provide a wireless pyro/piezo sensor system, which wirelessly transmits muscle movement signals from the patient to the PSG.

Furthermore, there is a need to provide a wireless pyro/piezo sensor system to operate under battery power for the duration of the sleep study.

Furthermore, there is a need to provide a wireless pyro/piezo sensor system, which is re-chargeable after use.

Furthermore, there is also a need to provide a wireless pyro/piezo sensor system to thereby yield the required phase relationship between respiratory airflow (inspiration and expiration) to final graphical indication of the polarized piezoelectric film sensor signals on PSG machine display.

Furthermore, there is a need to provide a wireless pyro/piezo sensor system, which is low cost, easy to use and easy to maintain.

It is accordingly an objective of the present subject matter to provide a wireless pyro/piezo sensor system especially constructed to meet such needs and that simultaneously transmits data and information to a PSG, sleep diagnostic or sleep therapy device regarding respiratory airflow, respiratory effort, muscle movements or tissue vibrations using a multitude of single wireless pyro/piezo sensors.

Sleep disorder diagnostic methods involve the collection of sensor data during a sleep study, preferably but not necessarily, conducted using a PSG machine. Especially in a sleep laboratory, setting up and maintaining the multitude of wires leading from the plural sensor to the PSG machine during the sleep study is cumbersome for the sleep technician, unreliable for the sleep physician and uncomfortable for the patient. In various examples, the sensor transducer does not require any bias or external power to operate.

During operation in a typical sleep diagnostic application, such as in a sleep laboratory or a patient's home, in accordance with examples of the present subject matter, a patient is fitted with a multitude of wireless pyro/piezo sensors. Sleep scientists, sleep physicians and sleep technicians use the sensor to detect and properly diagnose specific sleep disorders and diseases. These include abnormal respiratory events occurring in the upper airway of the patient whereby appropriate sleep therapy may be prescribed.

During operation to treat a sleep disorder, such as in a sleep laboratory or a patient's home, a patient is fitted with either a single or a multitude of wireless pyro/piezo sensors in conjunction with a sleep therapy device taking the place of a conventional PSG machine.

A wireless pyro/piezo sensor system according to various examples of the present subject matter is directed toward diagnosing or treating patients with sleep disorders. A multitude of wireless respiratory sensors for detecting airflow, respiratory effort, abdominal and chest sum, tissue vibration (e.g. snore), muscle movement are attached to patients during preparation for a sleep study in order to diagnose and ultimately treat undesired sleep behavior or conditions, including, but not limited to, obstructive sleep apnea, central sleep apnea, complex sleep apnea, snoring, restless leg syndrome (RLS), periodic limb movement (PLM), Bruxism (teeth grinding and clenching), sudden infant death syndrome (SIDS) and other neurological disorders not necessarily related to sleep. The sensors transmit biomedical data to various types and levels of PSG machines.

The manner in which therapy can be provided to the patient is fully described in provisional U.S. Provisional Patent Application Ser. No. 61/090,966, filed Aug. 22, 2008, and entitled “Apparatus and Method for a Therapeutic Central Nervous System Stimulation Controller”, and U.S. patent application Ser. No. 12/583,581, filed Aug. 21, 2009 and entitled “Closed Loop Neuromodulator”, the contents of each are hereby incorporated by reference in their entirety.

### Wireless Pyro/Piezo Sensor

Different wireless pyro/piezo sensors are designed to come in different sizes to accommodate large adult, medium adult, and small adult, pediatric, infant and neonatal patients.

Different wireless pyro/piezo sensor types comprise respiratory airflow, respiratory effort, tissue vibration, and muscle movement, sensors.

The wireless pyro/piezo sensor includes of a pyro/piezoelectric PVDF film transducer exhibiting pyro/piezoelectric properties. A set of wires connects the pyro/piezoelectric PVDF film transducer to a wireless pyro/piezo sensor transceiver. The wireless pyro/piezo sensor transceiver is affixed to the pyro/piezoelectric PVDF film transducer using a chemical compound material, such as EPOT-TEK® H20E available from Epoxy Technology, Inc. of Billerica, Md. The wireless pyro/piezo sensor transceiver receives control information from the wireless pyro/piezo sensor base station and in response transmits the sensor data via a wireless communication link.

A plurality of sensors may all communicate with a common wireless pyro/piezo sensor base station.

### Wireless Pyro/Piezo Sensor Transceiver

A wireless pyro/piezo sensor transceiver according to one example of the present subject matter comprises a battery, a wireless battery charger, a charge pump, an analog signal multiplexer, a signal filter, an analog to digital converter, a low power micro controller, a radio frequency transceiver and a radio frequency antenna.

A wireless pyro/piezo sensor base station circuit according to one example of the present subject matter is comprised of a radio frequency antenna, radio frequency transceiver, micro controller, digital to analog converter, analog signal de-multiplexer, analog signal filter, power supply and power source.

In various embodiments, a wireless communication link between the wireless pyro/piezo sensor transceiver and the wireless pyro/piezo sensor base station may be operating in the wireless medical telemetry service (WMTS) band in North America, and other unlicensed ISM bands providing a dedicated spectrum to ensure reliable link for sensor signal transmission. However, the wireless communication link between the wireless pyro/piezo sensor transceiver and the wireless pyro/piezo sensor base station may also be operating in other licensed or unlicensed North American or International frequency bands as found to be appropriate.

### Wireless Pyro/Piezo Sensor Base Station

A base station according to one example of the present subject matter works with a wireless pyro/piezo sensor having an integral wireless pyro/piezo sensor transceiver for transmitting and receiving sensor derived information to and from a base station which connects and relates, via a multitude of wires, the received wireless pyro/piezo sensor information to an attached PSG machine.

The wireless pyro/piezo sensor base station circuit comprises a radio frequency antenna, radio frequency transceiver, microcontroller, digital to analog converter, analog signal de-multiplexer, analog signal filter, power supply and a power source.

On one side, the wireless pyro/piezo sensor base station is hardwired to the polysomnograph (PSG) machine to further process the multitude of sensor signals and information. On the other side, the wireless pyro/piezo sensor base station transmits and receives wireless signals from a multitude of wireless pyro/piezo sensors equipped with integral wireless pyro/piezo sensor transceivers.

The following detailed description includes discussion of the configuration of the wireless pyro/piezo sensor system.

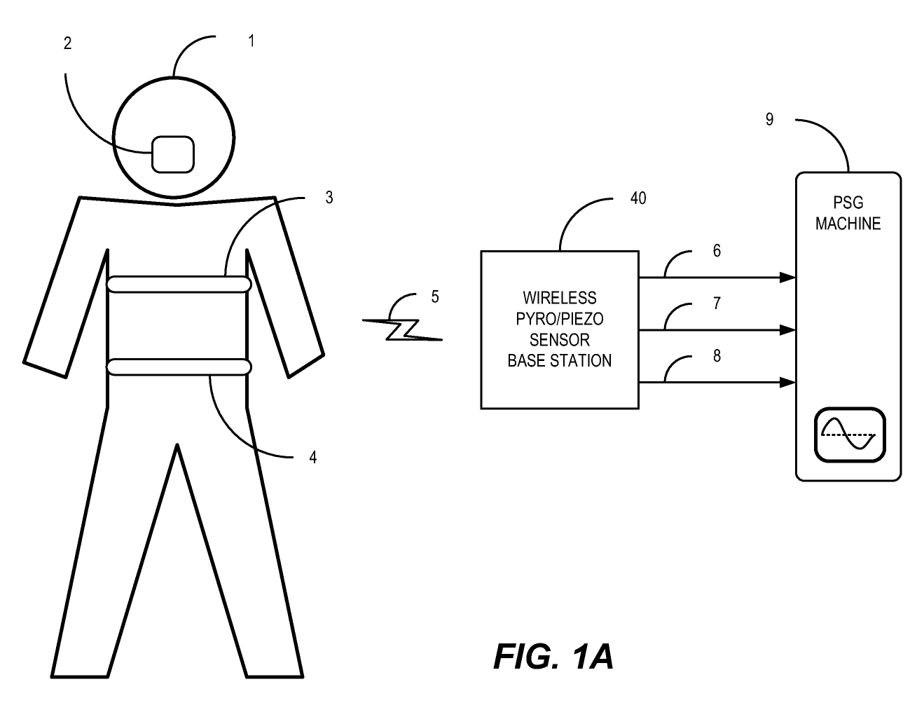

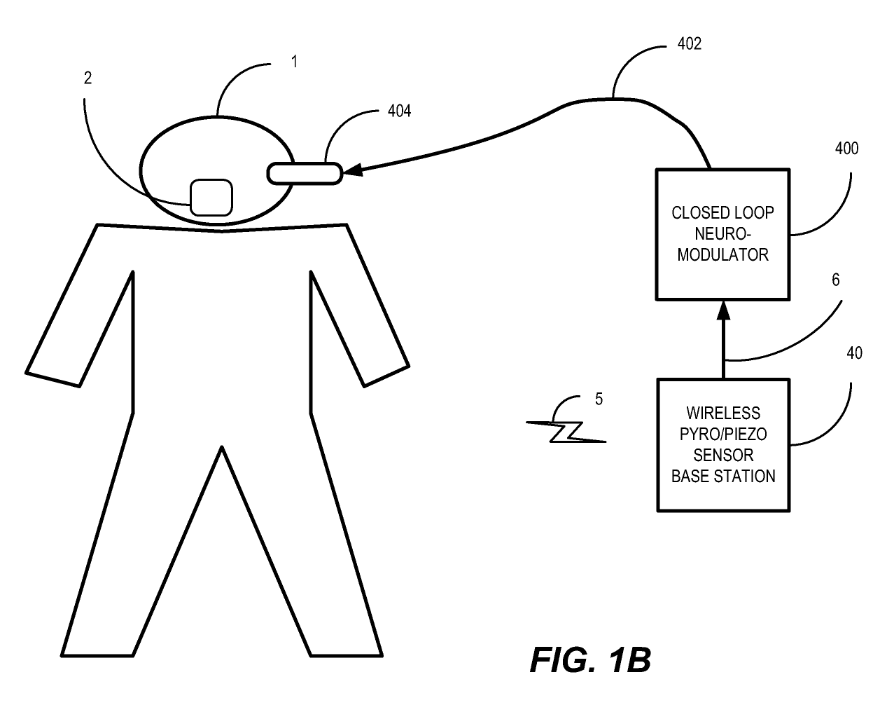

The present subject matter can be readily understood from FIGS. 1 through 8.

FIG. 1A shows an overall use and configuration of a wireless pyro/piezo sensor system according to one example of the present subject matter in a sleep diagnostic application. A typical sleep diagnostic patient 1 suffering from a sleep disorder has been outfitted with a sensor 2 to measure respiratory airflow, with a sensor 3 to measure chest effort and a sensor 4 to measure abdominal effort. A wireless communication link 5 connects the sensors to the input of the wireless pyro/piezo sensor base station 40. The output of the wireless pyro/piezo sensor base station 40 connects via a multitude of wires representing the multitude of sensor measurements. An airflow output 6, a chest effort output 7 and an abdominal effort output 8 connect, via a set of wire leads, to a conventional, commercially available sleep lab PSG machine 9.

FIG. 1B shows an overall use and configuration of a wireless pyro/piezo sensor system according to one example of the present subject matter in a sleep therapy application. A typical sleep therapy patient 1 suffering from a sleep disorder has been outfitted with a sensor 2 to measure respiratory airflow. A wireless communication link 5 connects the sensors to the input of the wireless pyro/piezo sensor base station 40. The output 6 of the wireless pyro/piezo sensor base station 40 connects to a closed loop neural modulator 400 as taught in the above-referenced provisional patent application U.S. File 61/090,966 filed on Aug. 22, 2008. The output of the closed loop neural modulator connects to a transducer 404 via connection 402 for the purpose of applying precise stimulation dosage signals to the patient's central nervous system, all as is fully explained in the above-mentioned provisional patent application.

In various embodiments, the wireless base station may couple more or less information to the PSG machine or closed loop neural modulator than that displayed in either of FIGS. 1A and 1B, including, but not limited to, airflow information, including respiration temperature and/or pressure information, chest respiratory effort information, abdominal respiratory effort information, snore information, tissue vibration information, muscle motion information or combinations thereof.

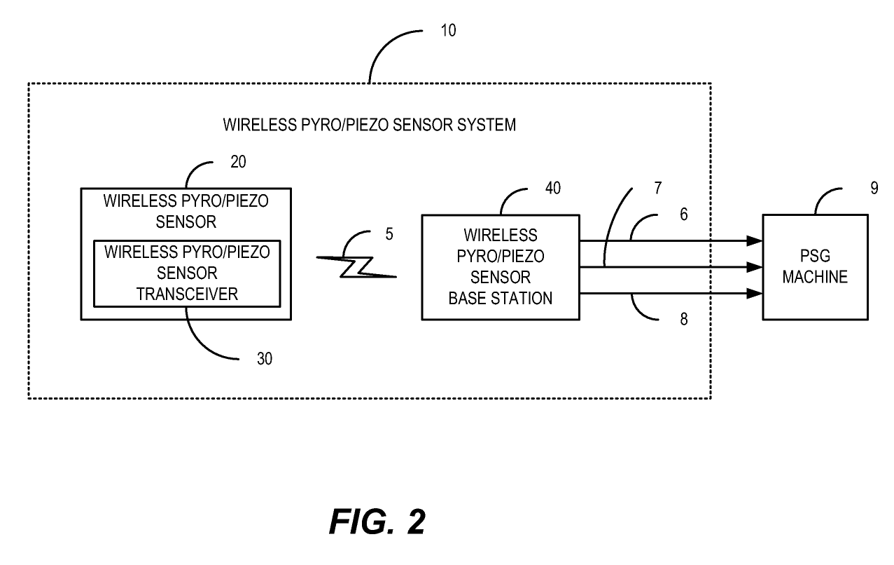

FIG. 2 shows elements and configuration of a wireless pyro/piezo sensor system 10 according to one example of the present subject matter. A wireless pyro/piezo sensor 20 with integrated wireless pyro/piezo sensor transceiver 30 transmits the wireless pyro/piezo sensor patient bio data, via communication link 5, to the wireless pyro/piezo sensor base station 40. The output of the wireless pyro/piezo sensor base station 40 connects, via a multitude of wires 6, 7 and 8 representing the multitude of sensed parameters being measured. An airflow output on wire 6, a chest effort output on wire 7 and an abdominal effort output on wire 8 connect to the sleep lab PSG machine 9.

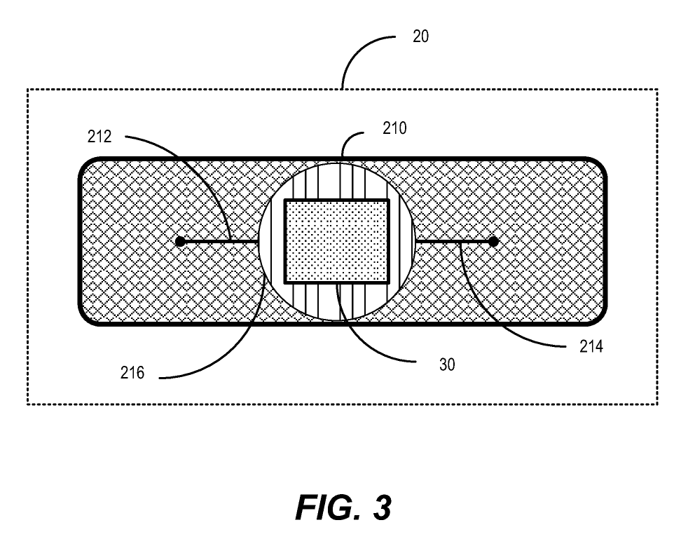

FIG. 3 shows a front view of an arrangement for a wireless pyro/piezo sensor 20 according to one example of the present subject matter. A wireless pyro/piezo sensor transceiver 30 is physically attached to the pyro/piezoelectric PVDF film transducer 210 via a suitable glue compound material 216 such as a die attachment compound like EPOT-TEK® H20E available from Epoxy Technology, Inc, of Billerica, Mass. The wireless pyro/piezo sensor transceiver is connected via wire terminal 212 and wire terminal 214 to the pyro/piezoelectric PVDF film transducer 210.

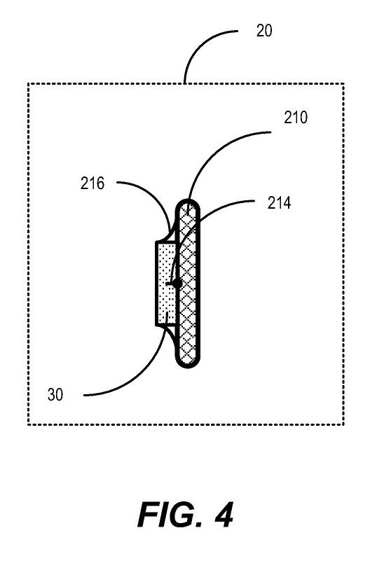

FIG. 4 shows a side view of the typical elements and configuration for a wireless pyro/piezo sensor 20 according to one example of the present subject matter. A wireless pyro/piezo sensor transceiver 30 is physically attached to the pyro/piezoelectric PVDF film transducer 210 via the glue compound material 216. The wireless pyro/piezo sensor transceiver is connected via wire terminal 214 (visible in this view only) to the pyro/piezoelectric PVDF film transducer 210.

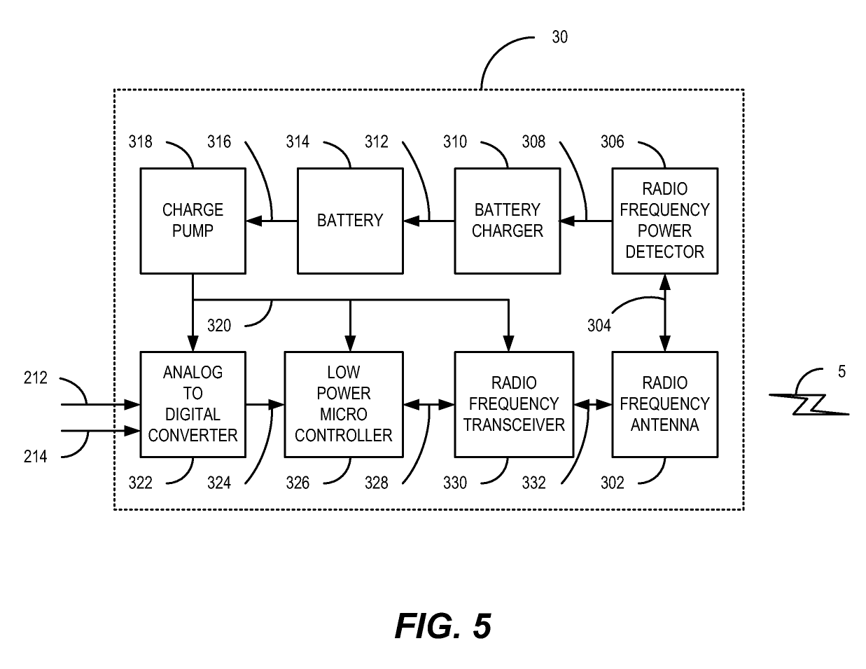

Referring to FIG. 5, there is indicated generally by numeral 30 a block diagram of a wireless pyro/piezo sensor transceiver, according to one example of the present subject matter, along with a wireless communication link 5. Attached to the wireless pyro/piezo sensor transceiver is the pair of wire terminations 212 and 214 which receive the pyro/piezoelectric PVDF film transducer output. Furthermore the wireless pyro/piezo sensor transducer indicated generally by numeral 30, contains a radio frequency antenna 302, connected via connection 304 for the purpose of charging and powering a radio frequency power detector 306. The radio frequency power detector 306 is connected via connection 308 to a battery charger 310 connected, via connection 312, to a battery 314. The battery charger 310 utilizes the detected RF energy from the radio frequency power detector 306 to charge the battery 314 which are available in various forms commercially. Battery 314 is connected, via connection 316, to a charge pump circuit 318 which provides power, via connection 320 to the analog to digital converter 322, a low power micro controller 326 and a radio frequency transceiver 330. The charge pump 318 functions as a voltage multiplier and those wishing to understand more fully the design and operation of such circuits are referred to “Charge Pump Circuit Design” by Feng & Japan, © 2006. McGraw-Hill Companies, Inc. (ISBN 0-07-147045-X).

The analog to digital converter 322 and the low power micro controller 326 are available as an integrated device in form of the C8051F350 from Silicon Laboratories of Austin, Tex.

The radio frequency transceiver 330 and the radio frequency antenna 302 are available as an integrated device in form of the RCT-AS and the RCR-RP from Radiotronix™ of Moore, Okla.

The battery 314, the battery charger circuit 310 and the radio frequency power detector circuit are available in from a thin film rechargeable battery CBC050 from Cymbet Corporation of Elk River, Minn.

The charge pump 318 is a classical buck-boost switch mode power supply topology and is available from in many different implementations from Texas Instruments of Dallas, Tex.

For the signal path, the pyro/piezoelectric PVDF transducer connections 212 and 214 are connected to the analog to digital converter 322 and the digital output therefrom is connected, via connection 324, to a low power micro controller 326 and to a radio frequency transceiver 330 by connection 328. A radio frequency antenna 302 is employed to transmit the received pyro/piezoelectric PVDF transducer information from wire terminals 212 and 214 via communication link 5 to the remote base station.

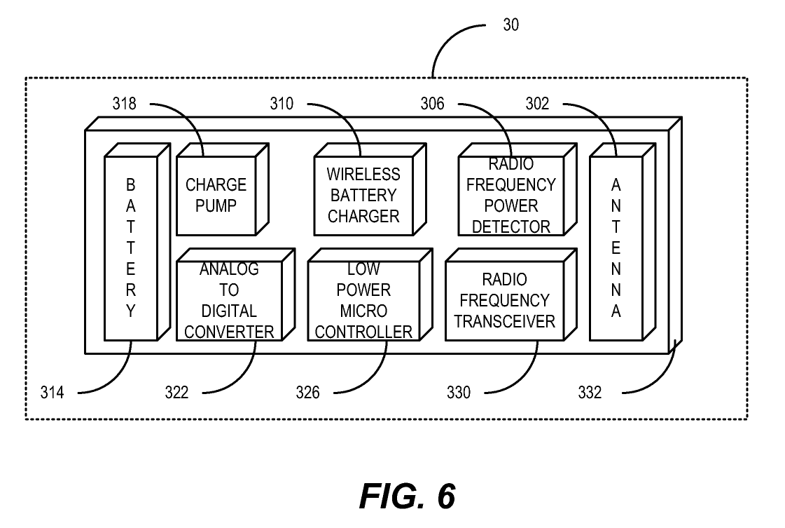

Referring to FIG. 6, there is indicated generally by numeral 30 an assembly layout of a wireless pyro/piezo sensor transceiver. Furthermore a wireless pyro/piezo sensor transducer indicated generally by numeral 30 contains a radio frequency antenna 302, a radio frequency power detector 306, a wireless battery charger 310, a battery 314, a charge pump 318, an analog to digital converter 322, a low power micro controller 326, a radio frequency transceiver 330.

Without limitation, the analog to digital converter 322 and the low power microcontroller 326 may be a C8051F350 available from Silicon Laboratories of Austin, Tex. The antenna 302 along with the radio frequency transceiver 330 may comprise a RCT-AS and the RCR-RP available from Radiotronix of Moore, Okla. Likewise, the battery 314 along with the battery charger circuit 310 and the RF power detector 306 are available from Cymbet Corporation of Elk River, Minn. The charge pump 318 is a classical buck-boost switch mode power supply topology and is available in several different implementations from the Texas Instruments Corporation of Dallas, Tex.

The silicon chip integration of FIG. 6 of the devices in die form of FIG. 5 is available in form of multi silicon die packaging from Crossfire Technologies of Eden Prairie, Minn.

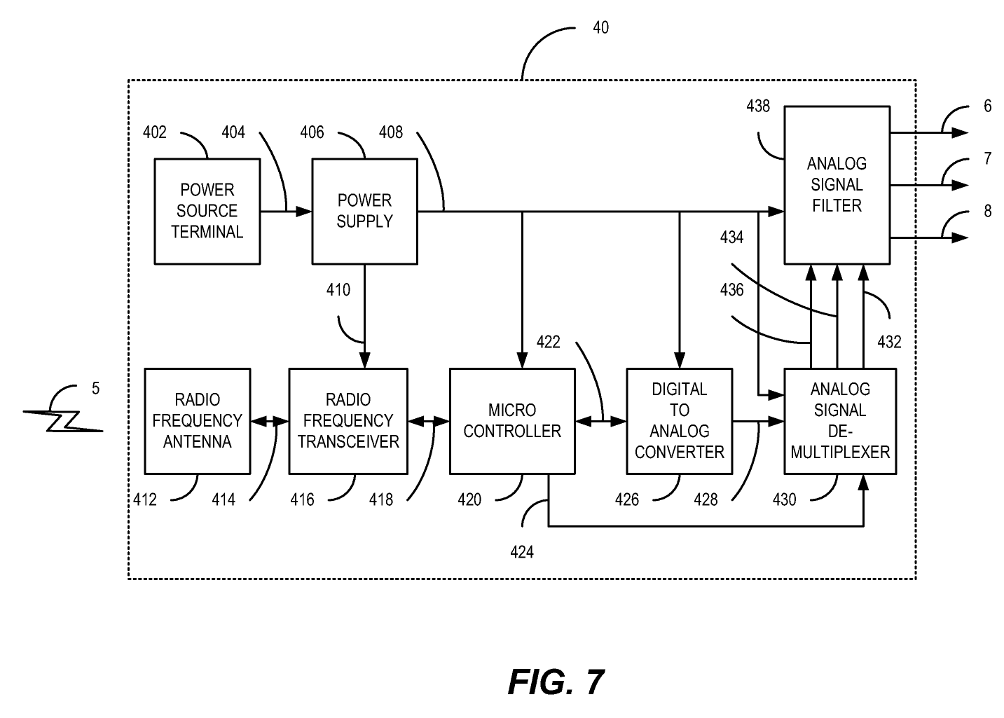

Referring to FIG. 7, there is enclosed by broken line box 40 a block diagram of a wireless pyro/piezo sensor base station, according to one example of the present subject matter, along with a wireless communication link 5. Attached to the wireless pyro/piezo sensor base station is a multitude of sensor signal output terminations 6, 7 and 8 which pass on the received wireless information from the multitude of wireless sensors to the attached PSG (as shown in FIG. 1). Furthermore the wireless pyro/piezo sensor base station contains a radio frequency antenna 412, connected via connection 414 to a radio frequency transceiver 416, such as Model RCT-AS and the RCR-RP, available from Radiotronix™ of Moore, Okla. Transceiver 416 is connected to a micro controller 420 by a connection 418. The microcontroller may be a Type C8051F350, available from Silicon Laboratories of Austin, Tex. and is connected via connections 422 and 424 to a digital to analog converter 426 and to an analog signal de-multiplexer 430 respectively. The signal de-multiplexer 430 preferably is part of the Type C8051F350, available from Silicon Laboratories of Austin, Tex. and is connected via connections 432, 434 and 436 to the analog signal filter 438. The analog signal filter 438 is a low pass device as taught in provisional patent application Ser. No. 61/123,781, filed Apr. 11, 2008 and entitled “APPARATUS AND METHOD FOR CREATING MULTIPLE POLARITY INDICATING OUTPUTS FROM TWO POLARIZED PIEZOELECTRIC FILM SENSORS” used to remove noise and connects via connections 6, 7 and 8 to the attached PSG machine. A power source terminal 402 which may be a 110 volt AC outlet connects via connection 404 to a power supply 406 that is, in turn, connected via output 408 to the micro controller 420, the digital to analog converter 426, and to the analog signal filter 438. Power supply 406 also supplies to a radio frequency transceiver, via connection 410 to provide appropriate biasing levels to those IC components.

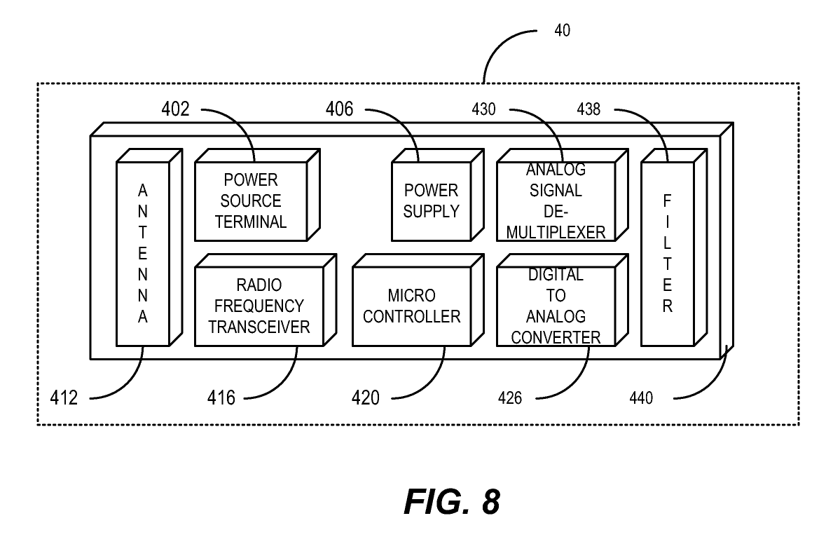

Referring to FIG. 8, there is indicated by a broken line box 40 an assembly layout of a wireless pyro/piezo sensor base station according to one example of the present subject matter. Furthermore, the wireless pyro/piezo sensor base station contains a radio frequency antenna 412, a power source terminal 402, a power supply 406, an analog signal de-multiplexer 430, an analog signal filter 438, a digital to analog converter 426, a microcontroller 420, and a radio-frequency transceiver 416.

The wireless pyro/piezo sensor system in FIG. 1 through 8 is based on the pyro/piezoelectric sensors constructed in accordance with the teachings of U.S. Pat. No. 5,311,875, U.S. Pat. No. 6,254,545, U.S. Pat. No. 6,485,432, U.S. Pat. No. 6,491,642, U.S. File No. 2007/0012089 and U.S. provisional application Ser. No. 61/075,136 Stasz, the teachings of which are hereby incorporated by reference as if fully set forth herein.

In various examples, the wireless pyro/piezo sensor system in FIG. 1 through 8 utilizes the respiratory effort belts made in accordance with the teachings of U.S. application Ser. No. 11/743,839, filed May 3, 2007 and entitled “Respiratory Sensing Belt Using Piezo Film” U.S. Pat. No. 6,894,427 respectively to Stasz, the teachings of which are hereby incorporated by reference as if fully set forth herein.

Those skilled in the art will understand and appreciate that various sensors are known including, but not limited to, thermocouples, thermistors, air pressure transducers, electrodes and respiratory effort belts and that these sensors are within the scope of the invention.

The present invention is advantageous because it does not require the sleep patient to be wired directly to the PSG machine during the sleep study. Fewer wire connections means greater patient comfort and mobility.

An additional advantage of the present invention is that it allows the collection of patient biomedical data from multiple wireless pyro/piezo sensors placed on a single patient on a non-interfering wireless sensor signal communication basis.

An additional advantage of the present invention is the collection of data from multiple wireless pyro/piezo sensors placed on a single patient without interfering with transmissions from other patients who may be present in the test facility.

An additional advantage of the present invention is that the wireless system allows for an indication of output polarity of its signal at the PSG thereby indicating that the respiratory signal changes are the result of either inspired or expired air movement.

An additional advantage of the present invention is the wireless transmission of respiratory airflow signals from the patient to the PSG machine.

An additional advantage of the present invention is the wireless transmission of respiratory effort signals from the patient to the PSG machine.

An additional advantage of the present invention is the wireless transmission of a chest effort and abdominal effort sum signals from the patient to the PSG machine.

An additional advantage of the present invention is the wireless transmission of tissue vibration signals from the patient to the PSG machine.

An additional advantage of the present invention is the wireless transmission of muscle movement signals from the patient to the PSG machine.

An additional advantage of the present invention is the wireless transmission of polarity indicating wireless pyro/piezo sensor output signals from the patient to the PSG machine.

An additional advantage of the present invention is the ability of the wireless pyro/piezo sensor to operate under battery power for the duration of the sleep diagnosis.

An additional advantage of the present invention is the ability of the wireless pyro/piezo sensor to operate under battery power for the duration of the sleep therapy.

An additional advantage of the present invention is the ability of the wireless pyro/piezo sensor's battery to be re-charged after use.

An additional advantage of the present invention is the ability to yield the required phase relationship between the respiratory airflow and effort (inspiration and expiration) to a graphical indication of the polarized piezoelectric PVDF film sensor signals on the display of the PSG machine.

An additional advantage of the present invention is that it is of low cost, easy to use and easy to maintain.

The present subject matter further supports the ability to provide a clean, safe, practical and convenient way to perform sleep diagnostics and sleep therapy in either a hospital sleep laboratory or in a home environment.

Examples of the present subject matter are described herein in considerable detail in order to comply with the patent statutes and to provide those skilled in the art with the information needed to apply the novel principles and to construct and use such specialized components. However, it is to be understood that the invention can be carried out by specifically different equipment and devices, and that various modifications, both as to the equipment and operating procedures, can be accomplished without departing from the scope of the invention itself.

The description of the various embodiments is merely exemplary in nature and, thus, variations that do not depart from the gist of the examples and detailed description herein are intended to be within the scope of the present disclosure. Such variations are not to be regarded as a departure from the spirit and scope of the present disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventor also contemplates examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system for detecting biomedical data for diagnosing sleep disorders of a patient, the system comprising:
a wireless sensor configured to detect the biomedical data from the patient;
a wireless base station configured to wirelessly receive the biomedical data from the wireless sensor; and
a polysomnograph (PSG) machine coupled to the wireless base station, the PSG machine configured to display information about the received biomedical data to a user for diagnosing sleep disorders.

2. The system of claim 1, wherein the wireless sensor is configured to produce a first output indicative of airflow temperature and to produce a second output indicative of airflow pressure.

3. The system of claim 1, including:
a processor coupled to the wireless base station, the processor configured to separate inspiratory and expiratory temperature change from the biomedical data; and
wherein the PSG machine is coupled to the processor and configured to display the separated inspiratory and expiratory temperature change information to the user for diagnosing sleep disorders.

4. The system of claim 3, wherein the PSG machine is configured to display the received biomedical data from the wireless sensor and the separated inspiratory and expiratory temperature change information from the processor to the user for diagnosing sleep disorders.

5. The system of claim 1, including:
a processor coupled to the wireless base station, the processor configured to separate inspiratory and expiratory pressure change from the biomedical data; and
wherein the PSG machine is coupled to the processor and configured to display the separated inspiratory and expiratory pressure change information to the user for diagnosing sleep disorders.

6. The system of claim 5, wherein the PSG machine is configured to display the received biomedical data from the wireless sensor and the separated inspiratory and expiratory pressure change information from the processor to the user for diagnosing sleep disorders.

7. The system of claim 1, including:
a processor coupled to the wireless base station, the processor configured to separate inspiratory and expiratory temperature change from the biomedical data and to separate inspiratory and expiratory pressure change from the biomedical data; and
wherein the PSG machine is coupled to the processor and configured to display the separated inspiratory and expiratory temperature change information and the separated inspiratory and expiratory pressure change information to the user for diagnosing sleep disorders.

8. The system of claim 7, wherein the PSG machine is configured to display the received biomedical data from the wireless sensor and the separated inspiratory and expiratory temperature change information and the separated inspiratory and expiratory pressure change information from the processor to the user for diagnosing sleep disorders.

9. The system of claim 1, wherein the wireless sensor includes at least one of a respiration sensor, a chest respiratory effort belt, or an abdominal respiratory effort belt.

10. The system of claim 1, wherein the wireless sensor includes a respiration sensor, a chest respiratory effort belt, and an abdominal respiratory effort belt.

11. The system of claim 10, wherein the wireless base station includes a single wireless base station configured to wirelessly receive biomedical data from each of the respiration sensor, the chest respiratory effort belt, and the abdominal respiratory effort belt.

12. The system of claim 1, wherein the wireless sensor includes at least one of a tissue vibration sensor or a muscle motion sensor.

13. A method for detecting biomedical data for diagnosing sleep disorders of a patient, the method comprising:
detecting biomedical data of the patient using a wireless sleep sensor;
wirelessly receiving the biomedical data from the sleep sensor using a wireless base station coupled to a PSG machine; and
displaying the sleep related information to a user using the PSG machine.

14. The method of claim 13, wherein the detecting biomedical data includes detecting respiratory airflow temperature and respiratory airflow pressure of the patient using a wireless airflow sensor.

15. The method of claim 13, including processing the biomedical data to separate inspiratory and expiratory temperature change information from the biomedical data; and
wherein the displaying includes displaying the separated inspiratory and expiratory temperature change information for diagnosing sleep disorders.

16. The method of claim 15, wherein the displaying includes displaying the received biomedical data and the separated inspiratory and expiratory temperature change information for diagnosing sleep disorders.

17. The method of claim 13, including processing the biomedical data to separate inspiratory and expiratory pressure change information from the biomedical data; and
wherein the displaying includes displaying the separated inspiratory and expiratory pressure change information for diagnosing sleep disorders.

18. The method of claim 17, wherein the displaying includes displaying the received biomedical data and the separated inspiratory and expiratory pressure change information for diagnosing sleep disorders.

19. The method of claim 13, including:
processing the biomedical data to separate inspiratory and expiratory temperature change from the biomedical data and to separate inspiratory and expiratory pressure change from the biomedical data; and
wherein the displaying includes displaying the separated inspiratory and expiratory temperature change information and the separated inspiratory and expiratory pressure change information to the user for diagnosing sleep disorders.

20. The method of claim 19, wherein the displaying includes displaying the received biomedical data, the separated inspiratory and expiratory temperature change information and the separated inspiratory and expiratory pressure change information to the user for diagnosing sleep disorders.

21. The method of claim 13, wherein the detecting biomedical data includes detecting at least one of chest respiratory effort information of the patient using a wireless chest respiratory effort belt, detecting abdominal respiratory effort information of the patient using a wireless abdominal respiratory effort belt, or detecting respiratory airflow information of the patient using a wireless pyro/piezoelectric airflow sensor.

22. The method of claim 13, wherein the detecting biomedical data includes detecting chest respiratory effort information of the patient using a wireless chest respiratory effort belt, detecting abdominal respiratory effort information of the patient using a wireless abdominal respiratory effort belt, and detecting respiratory airflow information of the patient using a wireless pyro/piezoelectric airflow sensor.

23. The method of claim 22, wherein the wirelessly receiving the biomedical data includes wirelessly receiving the biomedical data from the sleep sensor using a single wireless base station coupled to a PSG machine.

24. The method of claim 13, wherein the detecting biomedical data includes detecting biomedical data of the patient using at least one of a wireless tissue vibration sensor or a wireless muscle motion sensor.
